(19) United States
(12) Patent Application Publication
(10) Pub. No.: US 2020/0178805 A1
(43) Pub. Date: Jun. 11, 2020

(54) SYSTEMS AND METHODS FOR TARGETED BRAIN STIMULATION

(71) Applicant: Synaptec Network, Inc., Santa Monica, CA (US)

(72) Inventor: Sheldon Jordan, Pacific Palisades, CA (US)

(21) Appl. No.: 16/706,250

(22) Filed: Dec. 6, 2019

Related U.S. Application Data
(60) Provisional application No. 62/776,122, filed on Dec. 6, 2018.

(51) Int. Cl. A61B 5/00; A61B 5/055; A61B 5/16; A61B 5/12

(52) U.S. Cl. CPC A61B 5/0042; A61B 5/055; A61B 5/0048; A61B 5/165; A61B 5/4824; A61B 5/128; A61B 5/167

(57) ABSTRACT

Systems, methods, and devices for mapping brain activity to identify therapeutic targets in a patient are disclosed. A set of stimuli is applied to the patient, which includes stimulus that are at least one of provocative, soothing, or neutral with respect to a condition of a patient, for example addiction, phobia, disorder, etc. The patient focuses on one of the stimuli, in some embodiments suppressing the patient's mental or emotional response to the stimuli. As the patient responds to the stimuli, the patient's brain activity is monitored, for example by fMRI. After presenting a series of sets of stimuli to the patient and monitoring the patient's brain activity responsive to the stimuli, the brain activity is mapped to the condition based on the type of stimuli corresponding to the brain activity.

This application claims priority to U.S. provisional application 62/776,122, filed Dec. 6, 2018, the entire disclosure of which is incorporated herein by reference.

### FIELD OF THE INVENTION

The field of the invention is brain imaging.

### BACKGROUND

The background description includes information that may be useful in understanding the present invention. It is not an admission that any of the information provided herein is prior art or relevant to the presently claimed invention, or that any publication specifically or implicitly referenced is prior art.

The study and understanding of brain function is of keen interest in the art as it offers insights into the operation of the body, including overcoming obstacles or solving problems in day to day life as well as specific scenarios. For example, U.S. Pat. No. 8,265,743 to Aguilar, et al describes tracking eye movement in conjunction with electroencephalography (EEG) of targeted regions of an operator's brain to evaluate an operator's cognitive response to stimuli, for example potential danger in an open environment. While Aguilar appears to appreciate monitoring brain function to evaluate the operator's detection or identification of perceived danger or other stimuli, Aguilar does not appear to teach an analysis of what parts of the brain are experience increased activity in response to stimuli related to a condition.

All publications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

US Patent Publication No. 2012/0302868 to Kawashima attempts to monitor brain function using functional magnetic resonance imaging (fMRI) in response to visual texture stimulus, in order to evaluate the texture perception of a stimulus. While Kawashima attempts to indentify brain activity related to visual texture perception of a subject, it does not appear to teach using condition (e.g., fear, critical thinking, creativity, anxiety, depression, addiction, etc.) related stimuli to map brain activity linking the stimulus to the condition.

US Patent Publication No. 2016/0022168 to Luczak attempts to gauge brain plasticity of a patient as an indicator of whether a particular treatment or therapy is we suited for the patient at a given moment or under a specific circumstance. But Luczak apparently fails to appreciate identifying therapy targets in the brain by mapping brain function of a patient with a condition by introducing stimulus to the patient related to the condition.

WIPO Publication No. 2014/033588 to Karakas, et al teaches using fMRI to record a patient's brain pattern while introducing stimuli to a patient to induce inherent reaction or performance of a task, and training a subject to engage in such exercises. While Karakas also appears to teach specific regimens to map brain activity associated with specific conditions (e.g., attention deficient hyperactivity disorder (ADHD), etc.), it fails to teach relating the stimulus with the condition in order to improve mapping brain activity of a condition in a subject.

Thus, there is still a need for improved methods for mapping the brain activity of a patient associated with a condition through use of stimuli associated with the condition.

### SUMMARY OF THE INVENTION

The inventive subject matter provides apparatus, systems and methods in which a pattern of brain activity in a patient with a condition is mapped. A first set of stimuli is applied to the patient while a brain activity responsive to the first set of stimuli is monitored. A second set of stimuli is then applied to the patient while brain activity responsive to the second set of stimuli is also monitored. Using the first and second set of stimuli, and the respective monitored brain activities, the pattern of brain activity associated with the condition is mapped.

Methods and systems of preparing a patient for brain monitoring are also contemplated. Without monitoring the patient's brain, a first set of stimuli is applied to the patient and the patient does not suppress an emotional response to the first set of stimuli. Again, without monitoring the patient's brain, a second set of stimuli is applied to the patient and the patient suppresses an emotional response to the second set of stimuli. Typically, the first and second sets of stimuli include at least one of a tactile, an auditory, an aromatic, an electrical, or a visual stimulus related to a condition. In preferred embodiments, the first and second sets of stimuli comprise words that are either provocative (e.g., inflammatory) or neutral (e.g., unrelated) to the condition.

Systems and methods are also contemplated for mapping a pattern of brain activity in a patient to a cognitive function. Without monitoring the patient's brain, a set of stimuli is applied to the patient to illicit a gesture from the patient representing (or imitating) a stimulus in the set of stimuli. The set of stimuli is then applied to the patient to illicit a gesture from the patient representing (or imitating) the stimulus in the set of stimuli, this time while monitoring the patient's brain activity. A relation (e.g., provocative, neutral, etc.) between the set of stimuli and the cognitive function is used to map the pattern of brain activity to the cognitive function.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWINGS

FIG. 1 depicts a visual cue used in embodiments of the inventive subject matter.

FIG. 2 depicts a visual stimulus used in embodiments of the inventive subject matter.

FIG. 3 depicts another visual stimulus used in embodiments of the inventive subject matter.

FIG. 4 depicts a further visual stimulus used in embodiments of the inventive subject matter.

FIG. 5 depicts yet another visual stimulus used in embodiments of the inventive subject matter.

FIG. 6 depicts still another visual stimulus used in embodiments of the inventive subject matter.

FIG. 7 depicts yet a further visual stimulus used in embodiments of the inventive subject matter.

FIG. 8 depicts another visual stimulus used in embodiments of the inventive subject matter.

FIG. 9 depicts a further visual stimulus used in embodiments of the inventive subject matter.

FIG. 10 depicts yet another visual stimulus used in embodiments of the inventive subject matter.

FIG. 11 depicts still another visual stimulus used in embodiments of the inventive subject matter.

FIG. 12 depicts yet a further visual stimulus used in embodiments of the inventive subject matter.

FIG. 13 depicts another visual stimulus used in embodiments of the inventive subject matter.

FIG. 14 depicts a further visual stimulus used in embodiments of the inventive subject matter.

FIG. 15 depicts yet another visual stimulus used in embodiments of the inventive subject matter.

FIG. 16 depicts still another visual stimulus used in embodiments of the inventive subject matter.

FIG. 17 depicts a flow chart for an embodiment of the inventive subject matter to improve patient response to a procedure.

FIG. 18 depicts a flow chart for an embodiment of the inventive subject matter to map brain activity of a patient to a cognitive function.

FIG. 19 depicts a flow chart for an embodiment of the inventive subject matter to map brain activity of a patient related to a condition.

FIG. 20 depicts a flow chart for an embodiment of the inventive subject matter to map brain activity of a patient related to pain.

FIG. 21 depicts a flow chart for an embodiment of the inventive subject matter to map brain activity of a patient related to audible tone.

### DETAILED DESCRIPTION

The inventive subject matter provides apparatus, systems and methods in which a pattern of brain activity in a patient with a condition is mapped. A first set of stimuli is applied to the patient while a brain activity responsive to the first set of stimuli is monitored. A second set of stimuli is then applied to the patient while brain activity responsive to the second set of stimuli is also monitored. Using the first and second set of stimuli, and the respective monitored brain activities, the pattern of brain activity associated with the condition is mapped.

In some embodiments a response to the first set of stimuli is unsuppressed (e.g., experienced, embraced, explored, contemplated, considered, etc.) by the patient and, optionally, a response to the second set of stimuli is suppressed (e.g., self coaching, self soothing, self coping, self distraction, diverted attention, etc.) by the patient. However, it is also contemplated that the first set of stimuli be suppressed and the second set of stimuli unsuppressed, that both the first and second set of stimuli be suppressed, or that both sets be unsuppressed. It is also contemplated that the first set of stimuli includes both provocative and neutral stimuli, and optionally soothing stimuli, with respect to the condition. For example, where the condition is addiction, the first set of stimuli can include the word “Pills” or the image of prescription pills as a provocative stimulus, and further include the word “Tree” or the image of a tree as a neutral stimulus. It is also contemplated that the second set of stimuli can also include provocative and neutral stimulus.

The first set of stimuli can be the same (preferably substantially the same) as the second set of stimuli, with the exception of one element of the sets of stimuli. Viewed from another perspective, the two sets of stimuli can be identical, except for example the first set is displayed on a white background while the second set is displayed on a black background, or other combinations (e.g., blue/yellow, red/cyan, green/magenta, etc.). Preferably, the sets of stimuli include stimulus that are tactile, auditory, visual, aromatic, thermal, electrical, or combinations thereof, though combinations of these stimuli are also contemplated. Though, in some embodiments the first and second sets of stimuli are exactly the same.

In some embodiments it can be favorable to train patients before brain monitoring is conducted. For example, a training regimen of applying the first and second sets of stimuli to the patient (and subsequent steps, such as un/suppressed, etc.) before the steps of applying stimuli and monitoring brain activity. Generally applying the first stimuli and monitoring brain activity occurs contemporaneously, or is at least substantially concurrent.

It is contemplated that conditions to be mapped include both ailments (e.g., addiction, obsessive compulsive disorder, general anxiety disorder, social anxiety disorder, pain, and tinnitus, etc.) as well as healthy or accelerated brain functions (e.g., creativity, problem solving, decision making, interpretation, comprehension, memorization, etc.), and that at least some of the stimulus in the first and second sets of stimuli evoke the condition.

For example, the first set of stimuli can include a first pain stimulus while the second set of stimuli comprises a second pain stimulus that is different (e.g., more painful, less painful, sharp pain, dull pain, heat pain, cold pain, chemical pain, etc.) than the first pain stimulus. Likewise, the first pain stimulus can be associated with a first visual stimulus while the second pain stimulus is associated with a second visual stimulus, different than the first visual stimulus. Similarly, the first set of stimuli can include sets of words (e.g. one word, set of two words, set of three words, set of four words, etc.), such that each word in a set of words is either provocative or neutral, or optionally soothing.

Moreover, the first set of stimuli can include a first auditory stimulus and the second set of stimuli includes at least one auditory stimulus that is different than the first auditory stimulus. In some embodiments, the first auditory stimulus is associated with a first visual stimulus while at least one different auditory stimulus is associated with a second visual stimulus, which is also different than the first visual stimulus. It is also contemplated that the first set of stimuli includes sets of words, and the patient responds to a word from the sets of words with a manual gesture (e.g., hand gesture) that represents or imitates the word. It should be appreciated that a stimulus or set of stimuli can further incorporated a combination of various types of stimuli as discussed herein.

Methods and systems of preparing a patient for brain monitoring are also contemplated. Without monitoring the patient's brain, a first set of stimuli is applied to the patient and the patient does not suppress an emotional response to the first set of stimuli. Again, without monitoring the patient's brain, a second set of stimuli is applied to the patient and the patient suppresses an emotional response to the second set of stimuli. Typically, the first and second sets of stimuli include at least one of a tactile, an auditory, an aromatic, a thermal, an electrical, or a visual stimulus related to a condition, or combinations thereof. In preferred embodiments, the first and second sets of stimuli comprise words that are either provocative (e.g., inflammatory) or neutral (e.g., unrelated) to the condition, or optionally soothing.

Systems and methods are also contemplated for mapping a pattern of brain activity in a patient to a cognitive function. Without monitoring the patient's brain, a set of stimuli is applied to the patient to illicit a gesture from the patient representing (or imitating) a stimulus in the set of stimuli. The set of stimuli is then applied to the patient to illicit a gesture from the patient representing (or imitating) the stimulus in the set of stimuli, this time while monitoring the patient's brain activity. A relation (e.g., provocative, neutral, soothing, etc.) between the set of stimuli and the cognitive function is used to map the pattern of brain activity to the cognitive function.

It should be appreciated that, in combination with the systems and methods described, portions of the patient's brain that are known to be related to a specific condition can be stimulated externally, for example through use of extra (or intra) cranial magnets. Likewise, additional stimulus can be provided to the patient, for example video clips, music, animal or nature sounds, human voice (e.g., impersonal (stranger's voice) or personal (e.g., mother's voice)), or combinations thereof.

Likewise, it should be appreciated that maps of brain activity that are produced from the methods and systems described can be used to design therapies or treatments for the specific patient, targeting specific regions in the patient's brain for therapy. For example, if the results show that regions of the patient's brain are substantially inactive in response to the stimuli, but those regions are generally active in patients without the condition, then treatments or therapies can be designed for the patient to increase activity in the underperforming regions of the brain, for example directed magnets or introducing localized drugs. Similarly, if the regions of the specific patient's brain are extra-active in response to the stimuli as compared to patients without the condition, then therapies can be designed to decrease activity in the over-performing regions of the patient's brain.

Moreover, comparisons between brain activities in the suppressed and unsuppressed states can identify regions of the brain for potential therapy or treatment. For example, if the results show that portions of the patient's brain had increased activity when the patient did not suppress the emotional response to the stimuli, as compared to the activity when the patient did suppress the emotional response, then those regions showing decreased activity in the suppressed state can be further targeted for treatment or therapy. Likewise, if regions of the patient's brain had greater activity in the suppressed state than when the patient did not suppress emotional response to the stimuli, then the regions with increased activity in the suppressed state can also be targeted for treatment or therapy.

In part, the inventive subject matter visualizes activity in brain regions that are particularly relevant to specific conditions, for example ailments such as addiction, obsessive compulsive contamination disorder or OCD, general anxiety disorder (GAD), social anxiety disorder (SAD), pain, and tinnitus, and the ability to overcome that condition (e.g., cravings for addiction, compulsion for OC contamination disorder or OCD, anxiety for GAD or SAD, pain for pain disorder, ringing ears for tinnitus, etc.).

### EXAMPLE 1

To observe activity in brain regions that are particularly relevant to the condition, patients are asked to engage in a simple task. Patients are presented with a slideshow that contains words/concepts that have been empirically shown to generate a response in those with the specific condition. While these words/phrases may not be exactly what an individual patient finds to provoke the condition on a day-to-day basis, the patients should engage with the phrases so that they feel some pang of the condition (e.g., craving, desire, or urge related to addiction; anxiety, fear, uneasiness, or apprehension for OC contamination disorder, OCD, GAD, or SAD; etc.). It is contemplated that the attempt to feel the pang of the condition activates those parts of the brain that help quantify and map the patient's emotional response.

Slides are shown to the patient that are the exact same as the ones the patient will see during the fMRI. The first time the patient is shown these slides the background will be black. This is referred to as the “Unsuppressed” Condition. The patients should experience any elicited emotions without trying to cope or help themselves feel better. The patients should allow their thoughts to continue despite any craving they may feel.

When the patients look at the screen, they will see a list of 3 words/phrases. In some slides the words will be neutral, for example “See Tree”. In other slides the words will be provocative (e.g., “Grasp Pill” for addiction, “Stand in Crowd” for OC contamination disorder, “Count Things” for OCD, “Become Burden” for GAD, “Give Speech” for SAD, etc.). The patient selects any one of these 3 phrases to focus on while it remains on the screen. Patients should engage with that phrase to the best of their ability. If the lists repeat, the patients should try to pick a word or phrase that they have not already used for the present condition.

For example, if the patient focuses on “See Tree,” the patient should try to envision a scenario in which they see a tree. The patient should incorporate all senses to experience the scene in their mind if they can. The patient should consider how they feel in that scene. Likewise, when the patient sees a provocative phrase, they should try to visualize a scenario either as they imagine it would happen or how it has happened to the patient already. During this condition the patient should let their emotions happen, and not try to soothe or self cope.

The administrator will pause the presentation from time to time to have the patient rate their emotional state on a scale from 0 to 10 where “10” is the most intense emotion ever.

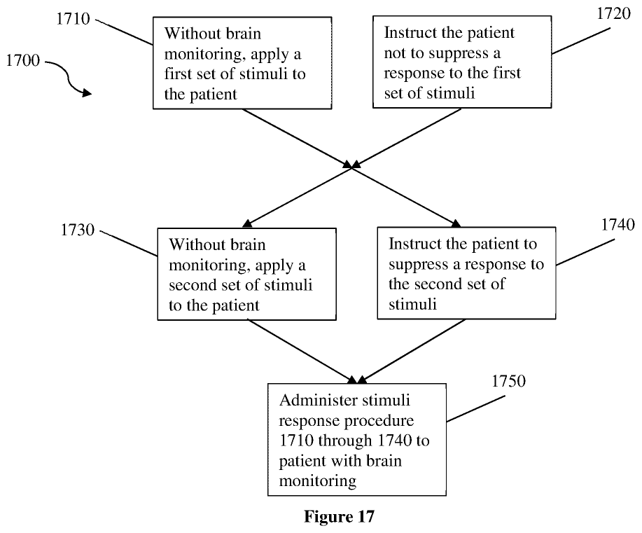

The patients will be shown the exact same slides a second time, but this time the slideshow will have a white background. This is referred to as the “Suppressed” condition. A key difference here is that once the patient has engaged with the provocative words, the patient should to try to talk themselves out of the pang of the condition that the patient may be feeling. The patient should try to think of positive ways of talking himself out of the negative emotional state. In some embodiments, the procedure of FIG. 17 is used to prepare the patient for the procedure and improve patient response.

For the addiction condition, a patient faced with a “Grasp Pill” concept can try to encourage himself: explain to himself that he is committed to ending his addiction; envision himself opening the trash can, throwing the pill away, and closing the lid; imagine himself then going on a long walk through the countryside or chatting with an old friend over coffee.

For the OC contamination disorder, if faced with a “Stand in Crowd” concept, the patient can try to encourage himself: explain to himself that he is okay, that his fears are out of proportion to the situation; explain to himself that he is healthy and will continue to be healthy, and standing in this crowd will not change that.

For the OCD condition, if faced with a “Count Things” concept, the patient can try to encourage himself: when the patient imagines a scenario where he begins counting objects, challenge any automatic thoughts he may have about it. Remind himself that it is unnecessary. Try to refocus his attention.

For the GAD condition, if faced with a “Lose Job” concept, the patient can try to encourage himself: explain to himself that he has a variety of skills and there is always important work waiting to be done; explain to himself that no matter what circumstances he has faced he has made it to today, and he can handle any hardship that may come.

For the SAD condition, if faced with a “Give Speech” concept, the patient can try to encourage himself: explain to himself that the audience is rooting for him; the patient has spent time preparing for this speech; this is not a life-or-death situation and, no matter how the speech goes, he will remain healthy.

The administrator should encourage the subject in the following way “we do not expect this to be easy and we also do not expect you to be successful. Remember that it is the sheer attempt that we are dependent on, not your success in regulating your emotions.”

The administrator will stop the session periodically to have the patient score the level of pang from the condition (e.g., craving, anxiety, fear, apprehension, etc.) from 0 to 10 where “10” is the worst ever.

When faced with the neutral group of words, the patient should relax. The patient can still engage with one of the 3 concepts, but do not try to talk yourself down. Simply visualize the neutral scenario as fully as possible.

This example can be further modified such that, in addition to suppressing or unsuppressing the emotion triggered by each stimuli, the patient also forms a hand gesture to imitate or represent each word that is focused on.

### EXAMPLE 2

To observe activity in brain regions that are particularly relevant to pain and overcoming pain, patients are asked to engage in a simple task. Using whatever apparatus is appropriate to stimulate their pain, the patient will be brought to a 7/10 pain threshold, 10 being the most pain the patient has felt in that region, 0 being none. This pain level will be measured and notated for the actual scan. During the stimulated phase during the scan, a technician will bring the patient to a 7/10, as notated during the training session, and then either increase or decrease the pain. The patient will then tap once if the pain increased or tap twice if the pain decreased. While uncomfortable, it is contemplated that the attempt to feel pain and anxiety activates those parts of the brain that help us quantify and map emotions.

Slides are shown to the patient that are the exact same as the ones the patient will see during fMRI. When the patient looks at the screen, the screen will have a (+) sign of varying colors. In some slides, the (+) sign will be white with a non-white (e.g., black) background, and nothing will happen during this slide. In other slides, the (+) sign will be green, this is the control and any stimulated pain will be reduced to a minimal level. In other slides, the (+) sign will be red, and the patient's pain will be stimulated to a 7/10, and then either increased above the 7/10 threshold or decreased below the 7/10 threshold.

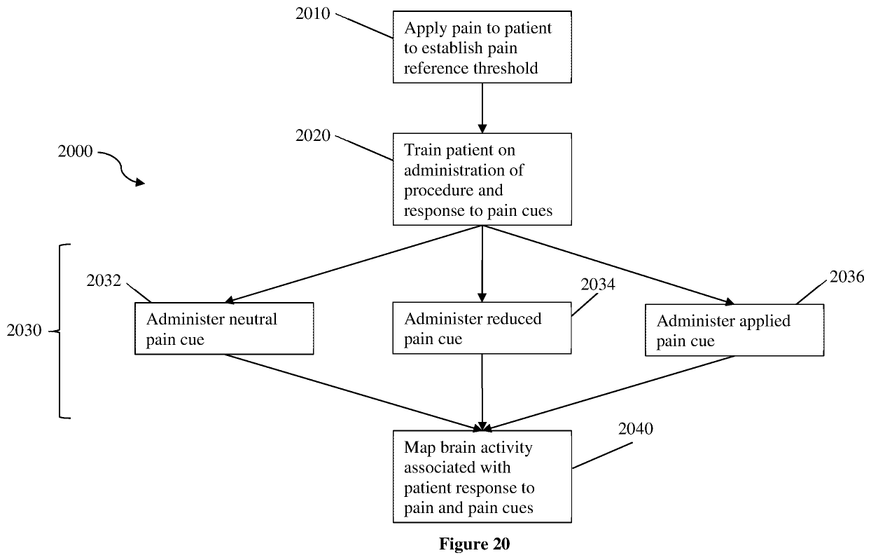

The patient should pay attention to the severity of the pain for any changes. When the (+) sign is red, after the patient is brought to a 7/10 threshold, the technician will either increase pain stimulation or decrease it. If the patient's pain is increased beyond the 7/10, the patient should tap once. If the patient's pain is decreased below the 7/10, the patient should tap twice. The patient should relax during the green (+) sign. Preferably, the patient will be in a comfortable position during the test, and the patient should tap/click with his dominant hand. FIG. 20 is representative of an embodiment of this type of procedure.

### EXAMPLE 3

To observe activity in brain regions that are particularly relevant to tinnitus and overcoming tinnitus, patients are asked to engage in a simple task. The patient is presented with a slideshow along with accompanying auditory tones, and then asked to make a decision regarding the frequency of one of the tones compared to its predecessor. While these tones may not be exactly what the patient personally find particularly provoking on a day-to-day basis, the patient should engage with the sounds as best they can.

Slides shown to the patient that are the exact same as the slides the patient will see during fMRI. The slides have (+) sign of varying colors. In some slides, the (+) is white (with non-white background, e.g., black) and there will be no sound. In other slides, the (+) sign is green and the patient will hear a single tone. In other slides, the (+) will be red and the patient will hear a series of tones while the (+) sign is red.

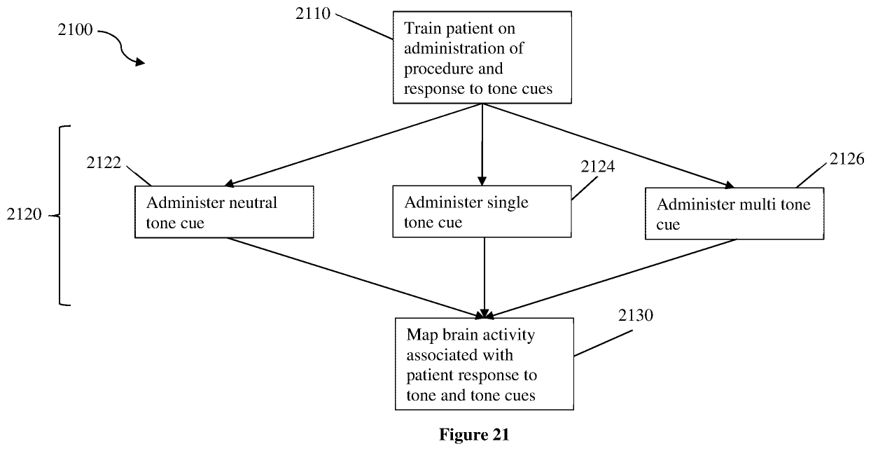

The patient should listen attentively to each sound for any changes. When the (+) sign is red, the patient will hear 3 tones. At the third tone during the red sequence, the patient should decide if that third tone is a higher frequency than the second tone in the red sequence. If the third tone is a higher frequency than the second tone, the patient should tap once. If the third tone is a lower frequency than the second tone, then the patient should tap twice. Preferably, the patients are in a comfortable position during the test, and tap/click with their dominant hand. FIG. 21 is representative of an embodiment of this type of procedure.

### EXAMPLE 4

To observe activity in brain regions that are particularly relevant to initiation of movement, making decisions, using working memory, and being imaginative or creative, patients are asked to engage in a simple task. Patients will be presented with a slideshow that contains several lists of words. Overall, the patient is asked to execute a particular hand motion that displays, simulates, or imitates how the word may look or act. Preferably, the patient should use their dominant hand for performing these motions, though it is contemplated that using both hands, or using the non-dominant hand may also provide relevant brain imaging data. It is contemplated that it is not the success of the patient in generating extremely creative motions that is clinically important. Rather, the sheer act of trying to be creative and deciding on a hand motion is generally enough to stimulate the relevant portions of the brain.

Slides are shown to the patient that will be the exact same as the ones the patient will see during fMRI. The patient should ignore the initial image with instructions. After the initial image, fMRI is used to monitor the patient's brain activity during the session. The patient will be presented with a series of slides, each containing three words. For each slide, the patient should choose one word and use their (preferably) dominant hand to represent or imitate that word. For example, if the patient chooses the word dog, the patient can use their hand to either represent what the dog looks like or how the dog might move. When the patient sees a slide with the (+) sign, the patient should relax and not move their hand. After a few minutes, the slides will repeat. When the patient sees a repeat slide, the patient should choose a word they did not use before, and again perform a hand motion the patient associates with that word.

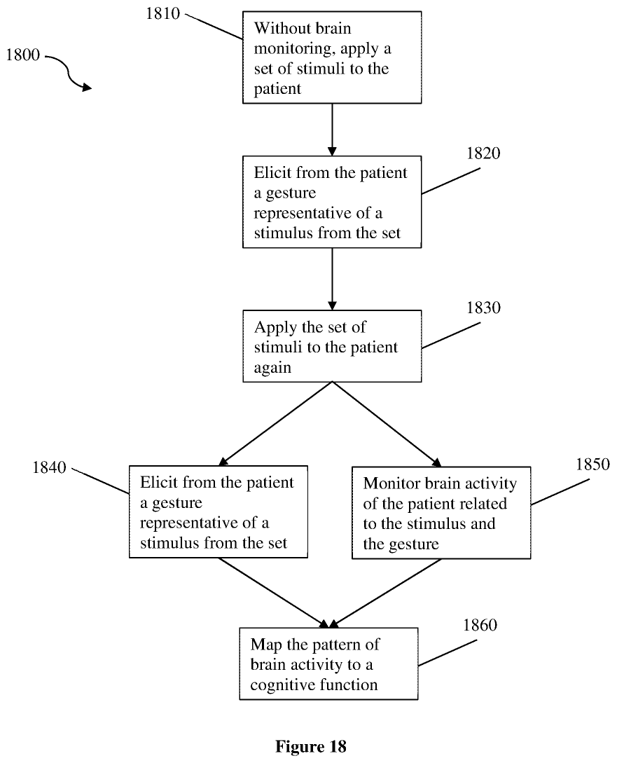

It is important that the patients minimize extreme motion; head motion may disrupt the scan quality. Preferably, the patient should find a comfortable position before starting the scan that allows them to move their fingers, and that the patient can hold for ˜10 minutes. The patient should not twist their forearms, and should restrict motion to the area from their wrist to their fingertips. FIG. 18 is representative of an embodiment of this type of procedure.

Modifications of this protocol include the patient further alternatively suppressing and unsuppressing stimuli in conjunction with performing hand gestures to simulate or represent each stimulus.

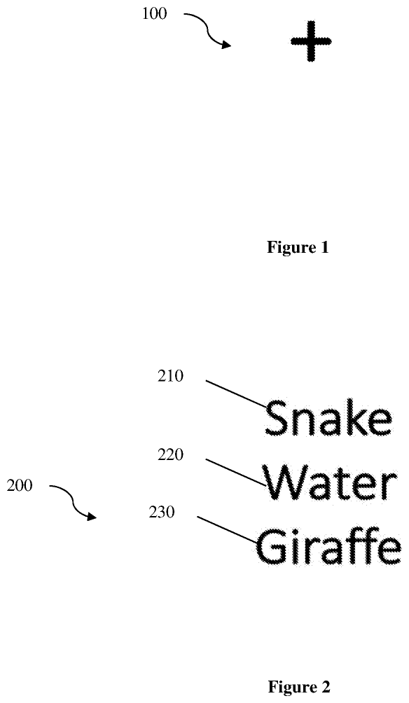

FIG. 1 depicts visual cue 100, which is shown to patients during procedures of the inventive subject matter. It can come in various colors and have various colored backgrounds, and cue the patient to steps in the procedures as described above.

FIG. 2 depicts visual stimulus 200, which is shown to patients during procedures of the inventive subject matter. Typically, the patient will select one word or phrase from among stimuli 210, 220, and 230 to use in the procedure as described in the examples above, for example imitate a hand gesture representative of the word. Alternatively, each of stimuli 210, 220, and 230 can be either neutral, provocative, or soothing to a condition of the patient. For example, stimulus 210 (Snake) may be provocative to a patient with a fear of snakes or reptiles, while stimuli 220 and 230 may be neutral to such a patient. Moreover, stimulus 220 (Water) may be provocative to a patient with a fear of drowning, while stimuli 210 and 230 are neutral. Similarly, stimuli 220 (Water) may be soothing to a patient with a fear of fire, while stimuli 210 and 230 are neutral. Typically FIG. 1 will follow FIG. 2 for one second or less.

FIG. 3 depicts visual stimulus 300 shown to patients during procedures of the inventive subject matter. Typically, the patient will select one word or phrase from among stimuli 310, 320, and 330 to use in the procedure as described in the examples above, for example imitated a hand gesture representative of the word, or as a neutral, provocative, or soothing stimulus. Typically FIG. 1 will follow FIG. 3 for one second or less.

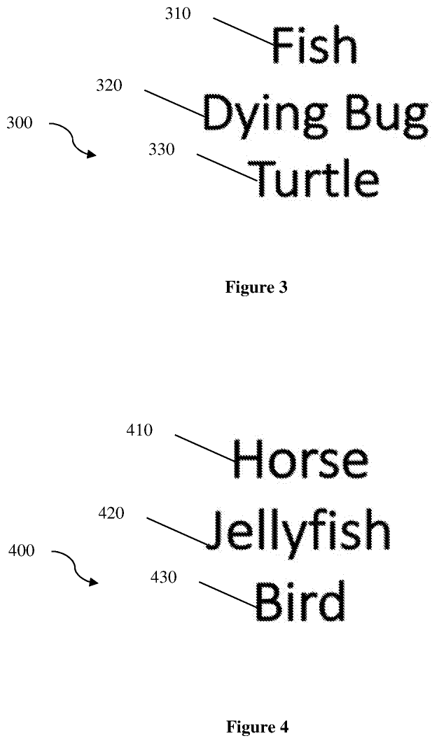

FIG. 4 depicts visual stimulus 400 shown to patients during procedures of the inventive subject matter. Typically, the patient will select one word or phrase from among stimuli 410, 420, and 430 to use in the procedure as described in the examples above, for example imitated a hand gesture representative of the word, or as a neutral, provocative, or soothing stimulus. Typically FIG. 1 will follow FIG. 4 for one second or less.

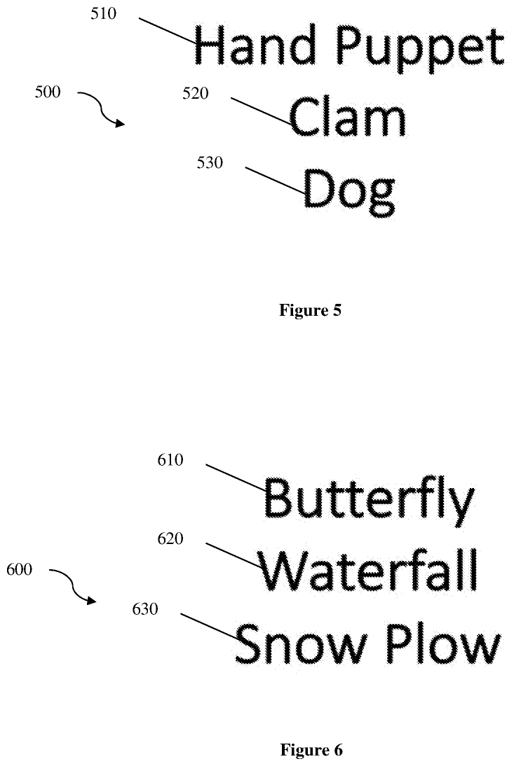

FIG. 5 depicts visual stimulus 500 shown to patients during procedures of the inventive subject matter. Typically, the patient will select one word or phrase from among stimuli 510, 520, and 530 to use in the procedure as described in the examples above, for example imitated a hand gesture representative of the word, or as a neutral, provocative, or soothing stimulus. Typically FIG. 1 will follow FIG. 5 for one second or less.

FIG. 6 depicts visual stimulus 600 shown to patients during procedures of the inventive subject matter. Typically, the patient will select one word or phrase from among stimuli 610, 620, and 630 to use in the procedure as described in the examples above, for example imitated a hand gesture representative of the word, or as a neutral, provocative, or soothing stimulus. Typically FIG. 1 will follow FIG. 6. In some embodiments, FIG. 1 will be displayed for a longer period than previously, for example more than 5, 10, or more seconds, indicating a transition from a training set to a monitoring set, or otherwise allowing the patient to relax between sets.

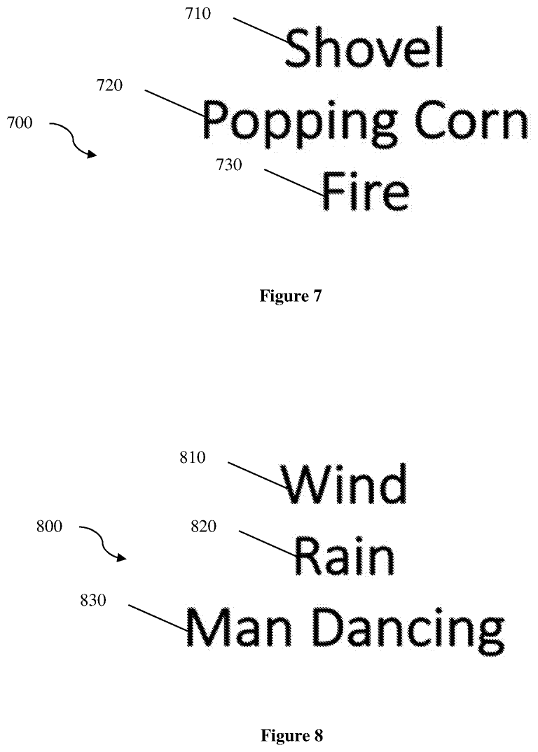

FIG. 7 depicts visual stimulus 700 shown to patients during procedures of the inventive subject matter. Typically, the patient will select one word or phrase from among stimuli 710, 720, and 730 to use in the procedure as described in the examples above, for example imitated a hand gesture representative of the word, or as a neutral, provocative, or soothing stimulus. Typically FIG. 1 will follow FIG. 7 for one second or less.

FIG. 8 depicts visual stimulus 800 shown to patients during procedures of the inventive subject matter. Typically, the patient will select one word or phrase from among stimuli 810, 820, and 830 to use in the procedure as described in the examples above, for example imitated a hand gesture representative of the word, or as a neutral, provocative, or soothing stimulus. Typically FIG. 1 will follow FIG. 8 for one second or less.

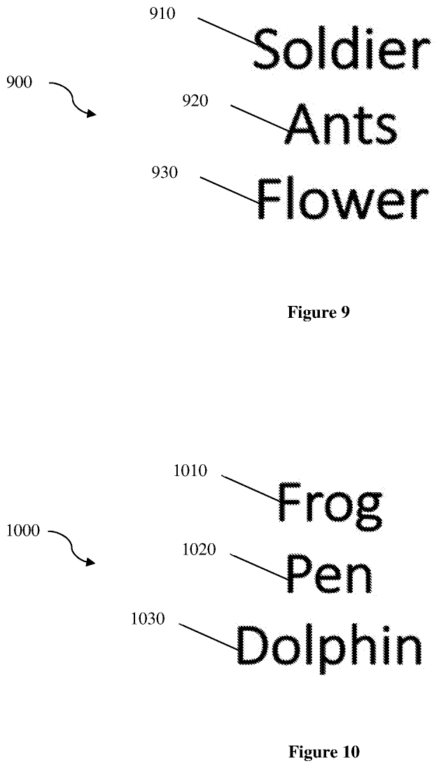

FIG. 9 depicts visual stimulus 900 shown to patients during procedures of the inventive subject matter. Typically, the patient will select one word or phrase from among stimuli 910, 920, and 930 to use in the procedure as described in the examples above, for example imitated a hand gesture representative of the word, or as a neutral, provocative, or soothing stimulus. Typically FIG. 1 will follow FIG. 9 for one second or less.

FIG. 10 depicts visual stimulus 1000 shown to patients during procedures of the inventive subject matter. Typically, the patient will select one word or phrase from among stimuli 1010, 1020, and 1030 to use in the procedure as described in the examples above, for example imitated a hand gesture representative of the word, or as a neutral, provocative, or soothing stimulus. Typically FIG. 1 will follow FIG. 10 for one second or less.

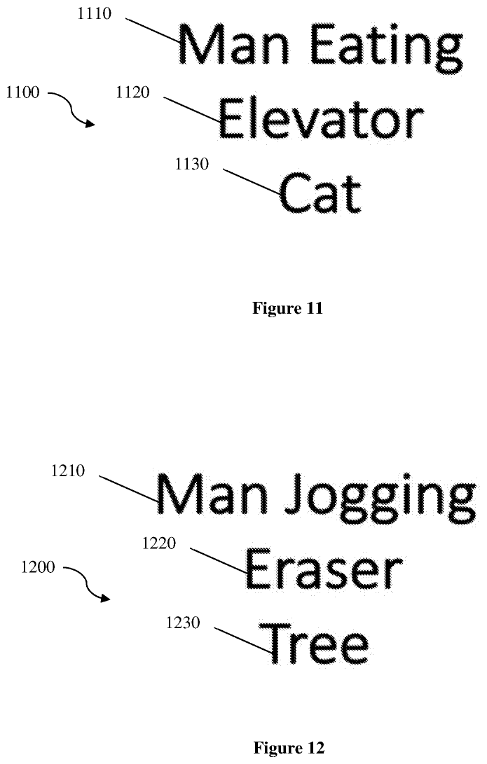

FIG. 11 depicts visual stimulus 1100 shown to patients during procedures of the inventive subject matter. Typically, the patient will select one word or phrase from among stimuli 1110, 1120, and 1130 to use in the procedure as described in the examples above, for example imitated a hand gesture representative of the word, or as a neutral, provocative, or soothing stimulus. Typically FIG. 1 will follow FIG. 11 for one second or less. In some embodiments, FIG. 1 will be displayed for a longer period than previously, for example more than 5 or 10 seconds, indicating a transition from a training set to a monitoring set, or to otherwise let the patient relax.

FIG. 12 depicts visual stimulus 1200 shown to patients during procedures of the inventive subject matter. Typically, the patient will select one word or phrase from among stimuli 1210, 1220, and 1230 to use in the procedure as described in the examples above, for example imitated a hand gesture representative of the word, or as a neutral, provocative, or soothing stimulus. Typically FIG. 1 will follow FIG. 12 for one second or less.

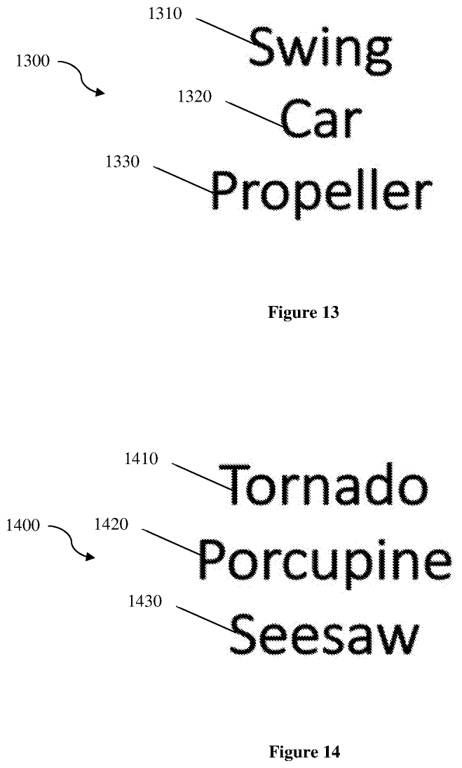

FIG. 13 depicts visual stimulus 1300 shown to patients during procedures of the inventive subject matter. Typically, the patient will select one word or phrase from among stimuli 1310, 1320, and 1330 to use in the procedure as described in the examples above, for example imitated a hand gesture representative of the word, or as a neutral, provocative, or soothing stimulus. Typically FIG. 1 will follow FIG. 13 for one second or less.

FIG. 14 depicts visual stimulus 1400 shown to patients during procedures of the inventive subject matter. Typically, the patient will select one word or phrase from among stimuli 1410, 1420, and 1430 to use in the procedure as described in the examples above, for example imitated a hand gesture representative of the word, or as a neutral, provocative, or soothing stimulus. Typically FIG. 1 will follow FIG. 14 for one second or less.

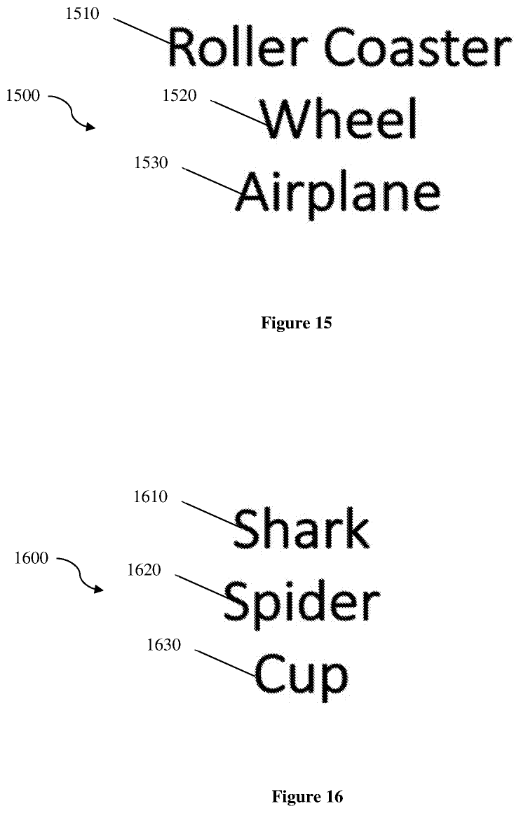

FIG. 15 depicts visual stimulus 1500 shown to patients during procedures of the inventive subject matter. Typically, the patient will select one word or phrase from among stimuli 1510, 1520, and 1530 to use in the procedure as described in the examples above, for example imitated a hand gesture representative of the word, or as a neutral, provocative, or soothing stimulus. Typically FIG. 1 will follow FIG. 15 for one second or less.

FIG. 16 depicts visual stimulus 1600 shown to patients during procedures of the inventive subject matter. Typically, the patient will select one word or phrase from among stimuli 1610, 1620, and 1630 to use in the procedure as described in the examples above, for example imitated a hand gesture representative of the word, or as a neutral, provocative, or soothing stimulus. Typically FIG. 1 will follow FIG. 16 for one second or less.

FIG. 17 is a flow chart for improving patient response to a procedure. Flow chart 1700 includes step 1710, where a first set of stimuli as described herein are administered to the patient. Any of the stimuli herein are appropriate, for example visual, tactile, auditory, olfactory, taste, or thermal. Step 1720 includes instructing the patient not to suppress a response to the first set of stimuli, and is typically concurrent with step 1710, or preferably shortly before step 1710. After steps 1710 and 1720, step 1730 includes applying a second set of stimuli to the patient, typically at least partially different than the first set. Step 1740 includes instructing the patient to suppress a response to the first set of stimuli, and is typically concurrent with step 1730, or preferably shortly before step 1730. It is critical to note that during both steps 1710 and 1730, the patient's brain activity is not monitored. After performing steps 1730 and 1740, step 1750 is performed, in which a stimuli response procedure is administered to the patient, during which the patient's brain activity is monitored, for example via fMRI.

FIG. 18 is a flow chart for mapping the brain pattern of a patient related to a cognitive function. Flow chart 1800 includes two training or preparation steps, 1810 and 1820. In step 1810, a set of stimuli as described herein is applied to the patient, followed by eliciting a gesture from the patient that is representative of a stimulus from the set, for example a hand, finger, arm, facial, mouth, or eye gesture, or some combination thereof. Steps 1810 and 1820 are performed without brain monitoring, as the objective of the procedure is to map brain activity related to forming the gesture, rather than brain activity related to learning a new task or being exposed to the procedure for the first time. However, it is contemplated that some procedures include monitoring the brain activity of the patient during the initial stimulus application and gesture formation, as it can be compared between subsequent procedures to distinguish between brain activity related to the initial exposure and brain activity from the patient when they are comfortable with the procedure.

Following the training steps, step 1830 again applies the set of stimuli to the patient. In steps 1840 and 1850, a gesture is elicited from the patient that represents one of the stimulus from the set, and the brain activity of the patient is monitored (e.g., fMRI) and recorded, respectively. Steps 1840 and 1850 are preferably performed contemporaneously, and in some embodiments the patient's brain activity is further monitored during at least a portion of step 1830. It is also contemplated that steps 1830, 1840, and 1850 can be repeated with additional sets of stimuli to develop a robust collection of monitored brain activity. Once the brain activity has been monitored and recorded, either a single time or over several repetitions, the recorded brain activity is compiled and mapped to the cognitive function performed by the patient in step 1860. For example, compared to baseline activity, areas of the brain showing increased or decreased activity in the patient while forming simulated gestures are associated with the cognitive functions of creativity, fine motor skills, abstract thought, or other cognitive functions.

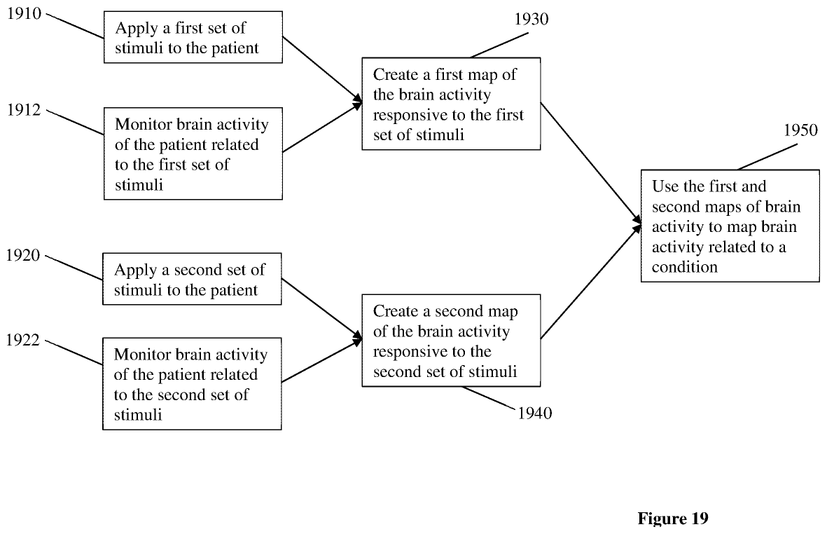

FIG. 19 depicts flow chart 1900 for mapping brain activity of a patient related to a condition. In steps 1910 and 1912, which preferably occur substantially contemporaneously, a first set of stimuli is applied to the patient and the brain activity of the patient responsive to the first set of stimuli is monitored and recorded, respectively. The stimuli are preferably related to the condition, though at least a portion of the stimuli can also be neutral or soothing of the condition. The recorded brain activity is used to create a first map of brain activity responsive to the first set of stimuli in step 1930. In steps 1920 and 1922, which preferably occur substantially contemporaneously, a second set of stimuli is applied to the patient and the brain activity of the patient responsive to the second set of stimuli is monitored and recorded, respectively. The recorded brain activity is used to create a second map of brain activity responsive to the second set of stimuli in step 1940.

While steps 1910 and 1912 precede step 1930, and likewise steps 1920 and 1922 precede step 1940, it is contemplated that steps 1910 and 1912, and steps 1920 and 1922, can be performed before or after each other, separated by 1, 5, 10, 15, or 20 minutes with or without interceding steps as described herein. For example, steps 1910 and 1912 can be performed, followed by steps 1920 and 1922, with steps 1930 and 1940 being performed after the completion of steps 1910, 1912, 1920, and 1922. Likewise, steps 1910 and 1912 can be performed followed by steps 1920 and 1922, with step 1930 being performed at least partially contemporaneously with steps 1920 and 1922. In such an embodiment, once steps 1920 and 1922 are complete, the brain activity map of step 1930 is also complete, and informs whether additional series of stimulus should be applied and monitored to develop a more robust collection of brain activity maps.

With steps 1930 and 1940 complete, the first and second maps of brain activity are used to map brain activity related to the condition in step 1950. In some embodiments, where the map of step 1950 is unsatisfactory, inconclusive, or requires further resolution, additional series of applying stimuli to the patient, monitoring brain activity, and mapping brain activity based on the stimuli are performed and further combined with the map of step 1950 to produce improved or refined brain activity maps related to the condition.

FIG. 20 depicts flow chart 2000 for mapping a patient's brain activity responsive to pain and pain cues. In step 2010, pain is applied and the patient's response (e.g., self-reported pain level, etc.) to the pain is recorded to establish a reference pain threshold. The patient is then trained on the administration of the procedure and responses to pain cues in step 2020. For example, the patient is trained that a neutral pain cue (e.g., visual, audible, etc.) indicates no pain will be applied, a reduced pain cue indicates any currently applied pain will be reduced, and an applied pain cue indicates the pain will be administered to the reference pain threshold. In some embodiments, once pain is brought to the reference pain threshold the pain is further increased or decreased from the threshold. In such embodiments, the patient is trained to make a response (e.g., tap finger, etc.) to whether the pain increases or decreases.

The pain cues are administered during stage 2030, including administration of neutral pain cues, reduced pain cues, and applied pain cues in steps 2032, 2034, and 2036, respectively. Steps 2032, 2034, and 2036 are performed in series, and can be randomly or intently arranged and repeated as required. Typically a pause of 5, 10, 15, 20, 30, or 45 seconds, or 1, 2, 3, 4, or 5 minutes separates each of steps 2032, 2034, and 2036. During stage 2030, and more preferably during each of steps 2032, 2034, and 2036, the patient's brain activity is monitored and recorded. Upon completion of stage 2030, the recorded brain activity is mapped and associated with the patient's responses to pain and the pain cues in step 2040. Where the map of step 2040 is inconclusive or otherwise insufficient, stage 2030 can be repeated and, optionally, parameters such as pause time between each of steps 2032, 2034, or 2036 can be modified, for example increased.

FIG. 21 depicts flow chart 2100 for mapping a patient's brain activity responsive to tone and tone cues. In step 2110, the patient is trained on the administration of the procedure and responses to tone cues. For example, the patient is trained that a neutral tone cue (e.g., visual, audible, etc.) indicates no tone will be played, a single tone cue indicates a single tone will be played, and multi tone cue indicates more than one tone will be played, at least one tone having a different pitch than the others. In some embodiments, the patient is trained to make a response (e.g., tap finger, etc.) to indicate whether the final tone played after a multi tone cue has a higher or lower pitch than the preceding tone.

The tone cues are administered during stage 2120, including administration of neutral tone cues, single tone cues, and multi tone cues in steps 2122, 2124, and 2126, respectively. Steps 2122, 2124, and 2126 are performed in series, and can be randomly or intently arranged and repeated as required. Typically a pause of 5, 10, 15, 20, 30, or 45 seconds, or 1, 2, 3, 4, or 5 minutes separates each of steps 2122, 2124, and 2126. During stage 2120, and more preferably during each of steps 2122, 2124, and 2126, the patient's brain activity is monitored and recorded. Upon completion of stage 2120, the recorded brain activity is mapped and associated with the patient's responses to the tones and the tone cues in step 2130. Where the map of step 2130 is inconclusive or otherwise insufficient, stage 2120 can be repeated and, optionally, parameters such as pause time between each of steps 2122, 2124, or 2126 can be modified, for example increased.

The following discussion provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

As used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

The following description includes information that may be useful in understanding the present invention. It is not an admission that any of the information provided herein is prior art or relevant to the presently claimed invention, or that any publication specifically or implicitly referenced is prior art.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

What is claimed is:

1. A method of mapping a pattern of brain activity in a patient to a condition, comprising:
applying a first set of stimuli to the patient and monitoring brain activity responsive to the first set of stimuli;
applying a second set of stimuli to the patient and monitoring brain activity responsive to the second set of stimuli; and
using the first and second set of stimuli and the respective monitored brain activity to map the pattern of brain activity to the condition.

2. The method of claim 1, wherein a response to the first set of stimuli is unsuppressed by the patient.

3. The method of claim 1, wherein a response to the second set of stimuli is suppressed by the patient.

4. The method of claim 1, wherein the first set of stimuli is the same as the second set of stimuli, with the exception of one element of the sets of stimuli.

5. The method of claim 1, wherein the sets of stimuli are selected from the group consisting of tactile, auditory, or visual stimuli.

6. The method of claim 1, further comprising a training regimen of applying the first and second sets of stimuli to the patient, wherein the training regimen is applied before the steps of applying and monitoring brain activity.

7. The method of claim 1, wherein the steps of applying the first stimuli and monitoring brain activity are substantially concurrent.

8. The method of claim 1, wherein the first set of stimuli comprises a provocative stimulus related to the condition, and a neutral stimulus.

9. The method of claim 1, wherein the first set of stimuli is the same as the second set of stimuli.

10. The method of claim 1, wherein the condition is selected from the group consisting of creativity, addiction, obsessive compulsive disorder, general anxiety disorder, social anxiety disorder, pain, and tinnitus, and at least one stimulus in the first set of stimulus is related to the condition.

11. The method of claim 1, wherein the first set of stimuli comprises a first pain stimulus and the second set of stimuli comprises a second pain stimulus different than the first pain stimulus.

12. The method of claim 11, wherein the first pain stimulus is associated with a first visual stimulus and the second pain stimulus is associated with a second visual stimulus different than the first visual stimulus.

13. The method of claim 1, wherein the first set of stimuli comprises sets of words, wherein each word in the sets of words is one of a provocative word related to the condition or a neutral word.

14. The method of claim 1, wherein the first set of stimuli comprises a first auditory stimulus and the second set of stimuli comprises at least one auditory stimulus different than the first auditory stimulus.

15. The method of claim 14, wherein the first auditory stimulus is associated with a first visual stimulus and the at least one auditory stimulus is associated with a second visual stimulus different than the first visual stimulus.

16. The method of claim 1, wherein the first set of stimuli comprises sets of words, and the patient responds to a word from the sets of words with a manual gesture representative of the word.

17. A method of preparing a patient for brain monitoring, comprising:
without brain monitoring, applying a first set of stimuli to the patient; and
without brain monitoring, applying a second set of stimuli to the patient;
wherein the patient does not suppress an emotional response to the first set of stimuli; and
wherein the patient suppresses an emotional response to the second set of stimuli.

18. The method of claim 17, wherein the first and second sets of stimuli comprise at least one of tactile, auditory, or visual stimuli related to a condition.

19. The method of claim 17, wherein the first and second sets of stimuli comprise words that are either provocative or neutral to a condition.

20. A method of mapping a pattern of brain activity in a patient to a cognitive function, comprising:
without brain monitoring, applying a set of stimuli to the patient to illicit a gesture from the patient representative of a stimulus in the set of stimuli;
applying the set of stimuli to the patient to illicit a gesture from the patient representative of the stimulus in the set of stimuli while monitoring the patient's brain activity; and
using a relation between the set of stimuli and the cognitive function to map the pattern of brain activity to the cognitive function.

21. The method of claim 20, wherein the patient suppresses an emotion evoked by the stimulus in conjunction with the gesture.

22. The method of claim 20, wherein the patient does not suppress an emotion evoked by the stimulus in conjunction with the gesture.
